(19) United States
(12) Patent Application Publication
(10) Pub. No.: US 2026/0226042 A1
(43) Pub. Date: Aug. 6, 2026

(54) CRYSTALLINE ACECLIDINE HCL HEMIHYDRATE AND PROCESSES FOR PREPARING THE SAME

(71) Applicant: OLON S.P.A., Rodano (MI) (IT)

(72) Inventors: Alessandro AGOSTI, Painesville, OH (US); Michael H. O'NEILL, Painesville, OH (US)

(73) Assignee: OLON S.P.A., Rodano (MI) (IT)

(21) Appl. No.: 19/151,688

(22) PCT Filed: Jan. 29, 2024
(86) PCT No.: PCT/IB2024/050788

§ 371 Date: Jul. 29, 2025

(30) Foreign Application Priority Data
Jan. 30, 2023 (IT) 102023000001386

(51) Int. Cl. C07D 453/02

(52) U.S. Cl. CPC C07D 453/02

(57) ABSTRACT

A crystalline Aceclidine HCl hemihydrate of formula I:

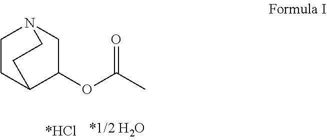

having a DSC onset peak at a value among 137.08° C. and 139.08° C. and/or a X-ray powder diffraction pattern with characteristic peak, expressed in 2-Theta values (2θ), at 17.7±0.2 and/or 20.47±0.2.

### TECHNICAL FIELD

The present invention relates to a hemihydrate form of Aceclidine HCl having formula I:

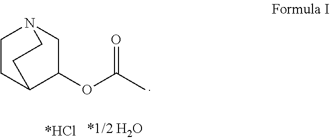

The invention further relates to processes for preparing hemihydrate form of Aceclidine HCl of formula I.

### BACKGROUND OF THE INVENTION

Aceclidine is known pharmaceutical active ingredient (API), particularly used in the ophthalmic medical field, which acts as a muscarinic acetylcholine receptor agonist. Aceclidine is a known parasympathomimetic miotic agent used in the treatment of narrow angle glaucoma. It decreases intraocular pressure.

US2016193193 (A1) discloses compositions comprising aceclidine and related methods for the treatment of presbyopia. Such compositions further comprise a polyol.

With particular reference to Aceclidine HCl, U.S. Pat. No. 3,997,543A discloses methods for the preparation thereof.

Moreover, Maria Kuhnert-Brandstätter & Friedrich Pröll (in article “Thermische Analyse von Hydraten organischer Verbindungen”, 1983), anhydrous and hydrate forms of Aceclidine are characterized.

In API manufacturing, a critical aspect is availability of stable forms, which are not subjected to undesired conversions into other forms. Generally, in order to avoid conversion into other forms, a common practice is to work under strictly controlled conditions (e.g. glove box).

For example, some anhydrous forms can absorb humidity resulting in a difficult handling if not under controlled conditions.

However, controlled conditions are not always applicable or convenient, so that it would be very advantageous to provide stable forms not subjected to undesired conversions.

This general technical problem also applies to Aceclidine, therefore there is the need of a stable form of Aceclidine which allows to easily handle such substance.

### SUMMARY OF THE INVENTION

The Applicant has now found a new crystalline form of Aceclidine, particularly Aceclidine HCl hemihydrate, which allows to advantageously overcome the above and other problems, further illustrated below.

Therefore, according to a first aspect, the present invention relates to a crystalline Aceclidine HCl hemihydrate as defined in claim 1.

According to a further aspect, the present invention relates to a process for preparing crystalline Aceclidine HCl hemihydrate as defined in claim 5.

According to a further aspect, the present invention relates to process for preparing crystalline Aceclidine HCl hemihydrate form of formula I as defined in claim 11.

Advantageously, Aceclidine HCl hemihydrate form of the invention is a stable form. Particularly, since this form has a low hygroscopicity (particularly being less hygroscopic compared to the corresponding anhydrous form), it does not require to be handled under controlled conditions. Indeed, unlike the corresponding anhydrous form, the form of the invention of formula (I) is not subjected to any conversion, so as working under controlled conditions (eg. glove box) is not required. Therefore, it is not necessary to establish humidity-controlled conditions. This is particularly relevant if we consider that, generally, the optimum values of relative humidity in workplaces would range between 40% and 60%. Such safety range is defined, for example, in the Italian law degree “Testo unico sulla Salute e Sicurezza sul Lavoro, Decreto Legislativo 81/2008”.

Moreover, pharmaceutical formulations based on Aceclidine are usually aqueous formulation, and for this reason it is important to obtain forms of Aceclidine stable in an aqueous environment.

Further aspects, characteristics and advantages of the invention will be more evident from the following detailed description.

### BRIEF DESCRIPTION OF THE FIGURES

FIG. 1 shows the XPRD diffractogram of Aceclidine HCl hemihydrate.

FIG. 2 shows the XPRD diffractogram of anhydrous Aceclidine HCl.

FIG. 3 shows the DSC of Aceclidine HCl hemihydrate.

FIG. 4 shows the DVS of Aceclidine HCl (Dynamic Vapor Sorption Isotherm).

### DETAILED DESCRIPTION OF THE INVENTION

For the scope of the present invention, in the following description and claims, definitions of numerical ranges comprise the single values within the range itself and the corresponding endpoints, unless otherwise specified.

For the scope of the present invention, in the following description and claims, the term “comprising” also includes the terms “consisting of” or “consisting essentially of”.

The crystalline Aceclidine HCl hemihydrate of formula I has a DSC onset peak at a value among 137.08° C. and 139.08° C. and/or a X-ray powder diffraction pattern with characteristic peak, expressed in 2-Theta values (2θ), at 17.7±0.2 and/or 20.47±0.2.

According to preferred a embodiment, the crystalline Aceclidine HCl hemihydrate of formula I has a DSC onset peak at a value among 137.08° C. and 139.08° C. or a X-ray diffraction pattern with characteristic peak, expressed in 2-Theta values (2θ), at 17.7±0.2 and/or 20.47±0.2.

According to a preferred embodiment, the crystalline Aceclidine HCl hemihydrate of formula I has a DSC onset peak at a value among 137.08° C. and 139.08° C. and a X-ray powder diffraction pattern with characteristic peak, expressed in 2-Theta values (2θ), at 17.7±0.2 and/or 20.47±0.2.

Preferably, the crystalline Aceclidine HCl hemihydrate of formula I has a X-ray powder diffraction pattern with an additional characteristic peak, expressed in 2-Theta values (2θ), at 28.84±0.2.

As demonstrated and evident from FIG. 4, the conversion of the anhydrous form of formula II to the hemihydrate form of formula I is irreversible. In fact, even when the environmental humidity content decreased the weight remained stable.

As already reported above, the invention also relates to a process for preparing crystalline Aceclidine HCl hemihydrate form of formula I comprising the step of converting the anhydrous form of formula II:

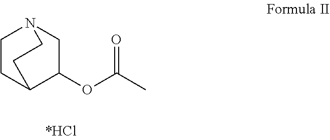

into Aceclidine HCl hemihydrate of formula I; wherein the conversion step is carried out by hydrating the anhydrous form of formula II.

According to a preferred embodiment, the hydration is carried out by insufflation of wet-air or wet-nitrogen.

According to an alternative preferred embodiment, the hydration is carried out by solubilization in water or in a mixture of water and a cosolvent. Preferably, the hydration is carried out until an environmental reaction humidity content of up to 16 wt %, more preferably up to 8 wt %, even more preferably between 4 wt & and 8 wt %, with respect to the weight of the starting anhydrous Aceclidine HCl form, is achieved.

Preferably, the hydration is carried out by solubilization in a mixture of water and a cosolvent, wherein the cosolvent is selected from esters and cyclic ethers. Preferably, the ester is selected from ethyl acetate and isopropyl acetate, more preferably is ethyl acetate. Preferably, the cyclic ether is selected from THF (tetrahydrofuran), methyl THE and dioxane.

As already reported above, the invention also relates to another process for preparing the crystalline Aceclidine HCl hemihydrate form of formula I. This alternative process comprises the step of converting the Aceclidine free base of Formula III:

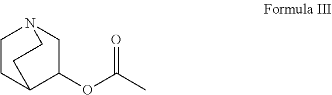

into Aceclidine HCl hemihydrate of formula I, wherein said step is carried out by adding HCl and water to a suspension of Aceclidine free base. Preferably, the water is added in an amount up to 16 wt %, preferably up to 8 wt %, more preferably between 4 and 8 wt %, with respect to the weight of the starting Aceclidine free base.

In such process, the solvent which can be used in order to suspend aceclidine free base is preferably selected from esters and cyclic ethers. Preferably, the ester ethyl acetate or isopropyl acetate, more preferably ethyl acetate. Preferably, the cyclic ether is selected from THE, methyl THE and dioxane.

The following examples are provided for illustrative purpose only, therefore no limiting interpretations to the scope of invention should be intended on the basis of such examples.

### Example 1: Hydration of Anhydrous Aceclidine HCl

The conversion from anhydrous to hemihydrate form of Aceclidine HCl (4 mg) was analyzed by a Dynamic Vapor Sorption (DVS) Isotherm. The observed conversion trend (weight change %/Relative humidity %) showed that the hemihydrate form formation is obtained at a weight change of about 4% (see FIG. 4), Such form clearly results to be irreversible even with a decreasing of relative humidity.

### Example 2: Synthesis of Aceclidine Free Base (Formula III)

A 500 ml jacketed reactor with overhead stirrer, equipped with nitrogen, reflux condenser was charged with 3-quinuclidinol (60 g), ethyl acetate (300 mL) and was stirred at a temperature of 20.0° C. to form a suspension. Then, acetic anhydride (68.4 g) was slowly charged over 10 minutes to vessel using a syringe. A clear solution was obtained. During this step; the temperature was raised from 20° C. to 30° C.

The bath temperature was adjusted to 53° C. and the solution stirred for 3 h (longer time if necessary) to get a complete reaction. After 3 h, an IPC was performed by GC-HP (90 μL of solution in 20 mL ethyl acetate) to check that 3-quinuclidinol was ≤0.5%. Then, the reaction mixture was cooled to room temperature and Aceclidine free base crystallized. The product was filtered through Chemrus disposable funnel 60 mL. The panel was rinsed with ethyl acetate (21.6 mL). Finally, the product solution (409.5 g) was charged back to the reactor.

### Example 3: Synthesis of Aceclidine HCl Hemihydrate (Formula I)

Aceclidine free base solution of formula III (137 g) previously obtained was further diluted with ethyl acetate (40 ml) and the bath temperature was adjusted to 17° C. Then, HCl gas (6.3 g) was bubbled maintaining an internal temperature lower than 30° C., After the charging of HCl, the temperature decreased. When the internal temperature reached a value less than 20° C., H₂O was charged at a concentration 8 wt % (2.6 g) with respect to the theoretical 100% yield of Aceclidine HCl salt. After the precipitation of the product, the mass of reaction was then stirred at 17° C. for 2 hours. The obtained solid was filtered through Chemrus disposable filter funnel 60 mL. The filtration was performed under nitrogen. The solid appeared as a very fine powder. The powder was washed with ethyl acetate (3 times×10 mb) under nitrogen. Finally, the solid was dried under vacuum at 50-55° C.; during step this the solid was grinded periodically.

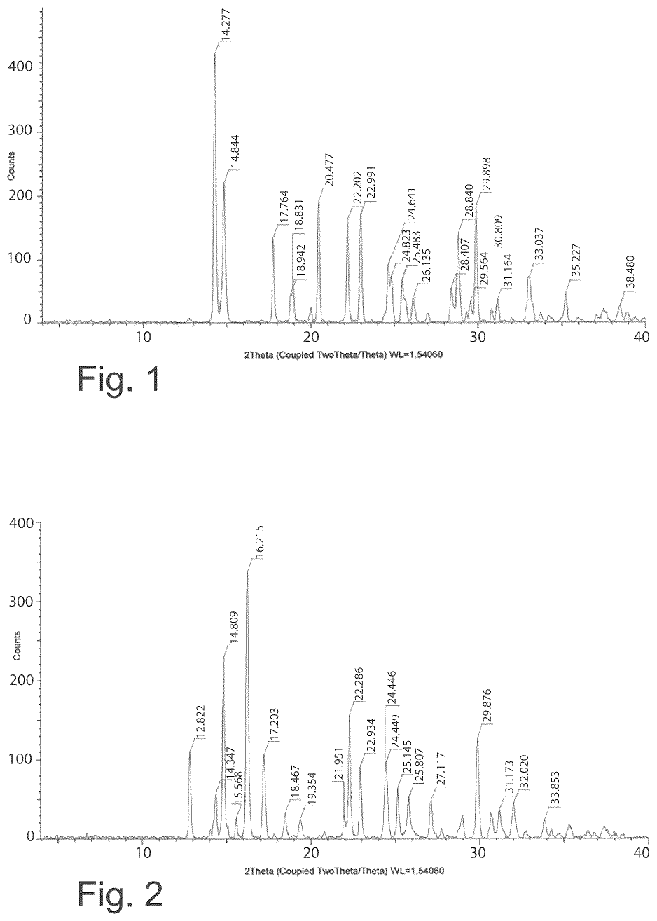

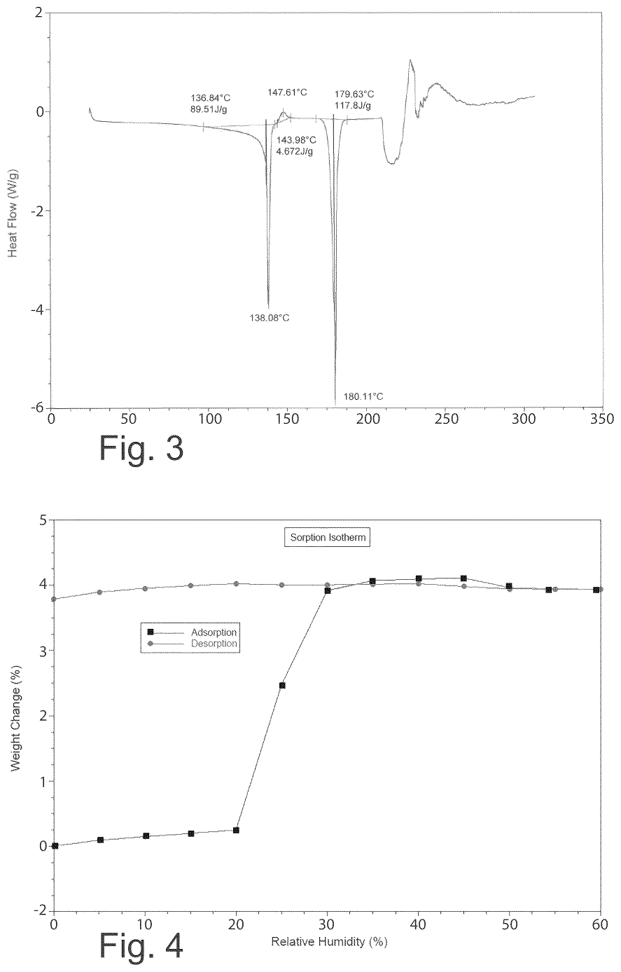

### Analysis 1: Pattern XPRD of the Hemihydrate Form of Aceclidine (Corresponding to FIG. 1)

TABLE 1

| | | | Rel. |
|---|---|---|---|
| Index | Angle | d Value | Intensity |
| 1 | 14.277 | 6.19886 | 100.0% |
| 2 | 14.844 | 5.96331 | 52.2% |
| 3 | 17.764 | 4.98905 | 31.6% |
| 4 | 18.831 | 4.70864 | 11.0% |
| 5 | 18.942 | 4.68130 | 15.2% |
| 6 | 20.477 | 4.33378 | 45.4% |
| 7 | 22.202 | 4.00081 | 38.2% |
| 8 | 22.991 | 3.86524 | 40.3% |
| 9 | 24.641 | 3.61002 | 21.7% |
| 10 | 24.823 | 3.58398 | 17.2% |
| 11 | 25.483 | 3.49257 | 16.2% |
| 12 | 26.135 | 3.40695 | 9.6% |
| 13 | 28.407 | 3.13936 | 13.0% |
| 14 | 28.840 | 3.09319 | 33.5% |
| 15 | 29.564 | 3.01908 | 8.0% |
| 16 | 29.898 | 2.98610 | 43.5% |
| 17 | 30.809 | 2.89985 | 4.9% |
| 18 | 31.164 | 2.86762 | 8.7% |
| 19 | 33.037 | 2.70922 | 17.0% |
| 20 | 35.227 | 2.54566 | 10.8% |
| 21 | 38.480 | 2.33762 | 6.4% |

### Analysis 2: Pattern XPRD of the Anhydrous Form of Aceclidine (Corresponding to FIG. 2).

TABLE 2

| | | | Rel. |
|---|---|---|---|
| Index | Angle | d Value | Intensity |
| 1 | 12.822 | 6.89882 | 32.8% |
| 2 | 14.347 | 6.16846 | 17.2% |
| 3 | 14.809 | 5.97724 | 68.1% |
| 4 | 15.568 | 5.68729 | 8.0% |
| 5 | 16.215 | 5.46181 | 100.0% |
| 6 | 17.203 | 5.15028 | 31.6% |
| 7 | 18.467 | 4.80059 | 9.7% |
| 8 | 19.354 | 4.58262 | 7.5% |
| 9 | 21.951 | 4.04587 | 9.1% |
| 10 | 22.286 | 3.98582 | 46.6% |
| 11 | 22.934 | 3.87465 | 27.2% |
| 12 | 24.446 | 3.63832 | 28.5% |
| 13 | 24.449 | 3.63796 | 28.5% |
| 14 | 25.145 | 3.53877 | 19.1% |
| 15 | 25.807 | 3.44950 | 15.7% |
| 16 | 27.117 | 3.28569 | 14.2% |
| 17 | 29.876 | 2.98833 | 37.8% |
| 18 | 31.173 | 2.86681 | 11.0% |
| 19 | 32.020 | 2.79291 | 13.3% |
| 20 | 33.853 | 2.64577 | 6.8% |

### The XPRD Analysis Method

Bruker D8 Discover X-ray Diffractometer
Vantec-500 Detector
Samples analyzed as received, R00099-50-27 was prepared in a glove box to limit moisture exposure.

### Detector Conditions

Voltage: 40 kV
Current: 40 mA
Radiation: Cu
Temperature: Ambient
X-ray source exit slit size: 0.5 mm pinhole
Snout collimator: 0.5 mm
Sample holder: ground quartz plate (R00099-50-21, R00099-50-24) or silicon disc in hermetic holder with mylar sheet cover (R00099-50-27).

### Operating Conditions

Detector Distance: 30.3 cm
Chi integration range: 4-40° 2⊖
Count time: 120 seconds/frame
#of frames: 3
Theta 1 position: 4 deg for quartz plate, 6 deg for silicon disc
Theta 2 position: 4 deg for quartz plate, 6 deg for silicon disc
Frame width: 12
Amplitude: 2 mm

### DSC Analysis Method

TA Instruments DSC Q2000
Samples analyzed as received.
2-10 mg weighed and added to Tzero aluminum pan then pressed with crimped lid
Equilibrated at 25° C.
Heated from 25-250° C. at 10° C./min.
Continuous nitrogen purge at 50 mL/min.

1. A crystalline Aceclidine HCl hemihydrate of formula I: 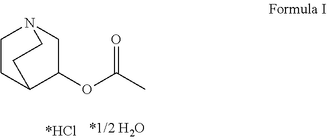
having a DSC onset peak of 137.08° C.-139.08° C. and/or an X-ray powder diffraction pattern with characteristic peak, expressed in 2-Theta values (2θ), at 17.7±0.2 and/or 20.47±0.2.

2. The crystalline Aceclidine HCl hemihydrate according to claim 1, having a DSC onset peak of 137.08° C.-139.08° C. or an X-ray powder diffraction pattern with characteristic peak, expressed in 2-Theta values (2θ), at 17.7±0.2 and/or 20.47±0.2.

3. The crystalline Aceclidine HCl hemihydrate according to claim 1, having a DSC onset peak of 137.08° C.-139.08° C. and an X-ray powder diffraction pattern with characteristic peak, expressed in 2-Theta values (2θ), at 17.7±0.2 and/or 20.47═0.2.

4. The crystalline Aceclidine HCl hemihydrate according to claim 1, having an X-ray powder diffraction pattern with an additional characteristic peak, expressed in 2-Theta values (2θ), at 28.84±0.2.

5. A process for preparing crystalline Aceclidine HCl hemihydrate form of formula I as defined in claim 1, comprising converting an anhydrous form of formula II 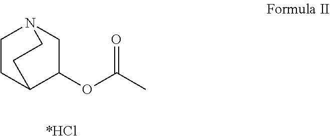
into Aceclidine HCl hemihydrate of formula I; wherein the conversion is carried out by hydrating the anhydrous form of formula II.

6. The process according to claim 5, wherein the hydration is carried out by insufflation of wet-air or wet-nitrogen.

7. The process according to claim 5, wherein the hydration is carried out by solubilization in water or a mixture of water and a cosolvent.

8. The process according to claim 7, wherein cosolvent is present and the cosolvent is selected from: esters and cyclic ethers.

9. The process according to claim 7, wherein the hydration is carried out until an environmental reaction humidity content of up to 16 wt %, with respect to the weight of the starting anhydrous Aceclidine HCl form, is achieved.

10. The process according to claim 9, wherein the environmental reaction humidity content is up to 8 wt % with respect to the weight of the starting anhydrous Aceclidine HCl form.

11. A process for preparing crystalline Aceclidine HCl hemihydrate form as defined in claim 1, comprising converting the Aceclidine free base of Formula III 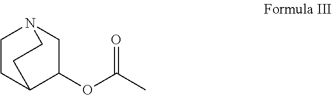
into Aceclidine HCl hemihydrate of formula I by adding HCl and water to a suspension of Aceclidine free base.

12. The process according to claim 11, wherein the water is added in an amount up to 16 wt % with respect to the weight of the starting Aceclidine free base.

13. The process of claim 11, wherein a solvent is used, said solvent being selected from esters.
